(19) United States
(12) Patent Application Publication
(10) Pub. No.: US 2006/0052727 A1
(43) Pub. Date: Mar. 9, 2006

(54) Activity monitoring device and weight management method utilizing same

(76) Inventor: Laurence Palestrant, Scottsdale, AZ (US)

Correspondence Address: Jordan M. Meschkow, Esq.;Meschkow & Gresham, PLC, Suite 409, 5727 North Seventh Street, Phoenix, AZ, 85014, US

(21) Appl. No.: 10/938,890

(22) Filed: Sep. 9, 2004

(51) Int. Cl. A61B 5/103; A63B 71/00; G08B 23/00

(52) U.S. Cl. 600/595; 340/573.1; 482/8

(57) ABSTRACT

An activity monitoring device (20) includes a movement sensor (44) for producing a movement signal (118) in response to an activity level of an individual. A stationary interval timer (38) determines a stationary interval (126) during which the movement signal (118) fails to exceed a movement intensity threshold (48). When the stationary interval (126) exceeds a stationary interval threshold (50), an indicator (54, 56) alerts the individual. The activity monitoring device (20) is worn by the individual and is utilized in a weight management method. In particular, the device (20) alerts the individual whenever the stationary interval (126) exceeds the stationary interval threshold (50) so that the activity level of the individual can be increased, for example, by fidgeting.

### TECHNICAL FIELD OF THE INVENTION

The present invention relates to the field of weight management in humans. More specifically, the present invention relates to an activity monitoring device for facilitating an awareness of, and increasing, an individual's activity level.

### BACKGROUND OF THE INVENTION

Obesity and overweight conditions are increasingly critical problems, particularly in the developed countries such as the United States. Indeed, an estimated one third of Americans are overweight, with an additional twenty-five percent being classified as obese. Being overweight significantly increases an individual's risk of developing a number of health conditions including type 2 diabetes, heart disease, stroke, colon cancer, post-menopausal breast cancer, endometrial cancer, gall bladder disease, osteoarthritis, obstructive sleep apnea, and such.

Overweight, obesity, and their associated health problems have a significant economic impact on the health care system and society, both directly and indirectly. Direct medical costs include, for example, preventative, diagnostic, and treatment services related to obesity. Indirect costs include, for example, the value of income lost from decreased productivity, restricted activity, absenteeism, and the value of future income lost by premature death. Accordingly, successful weight loss programs, exercise strategies, medications, and even surgery have become a large-scale industry.

Overweight and obesity are a result of energy imbalance over a long period of time. That is, fat gain occurs when more calories are consumed than are burned. There are three main factors involved in the burning of calories. These factors are basal metabolic rate (BMR), the burning of energy when the body is at rest; postprandial thermogenesis, the burning of energy in the digestion, absorption, and storage of food in the body; and physical activity, including exercise activities and nonexercise activity thermogenesis (NEAT).

Through subjective observations, it appears that some individuals are able to resist fat gain, even when they are overeating, whereas others readily store excess fat. A study conducted at the Mayo Clinic, in Rochester, Minn. attempted to identify the thermogenic factor that allows some individuals to resist weight gain despite overeating. An article entitled “Role of Nonexercise Activity Thermogenesis in Resistance to Fat Gain in Humans, Levine et al., Science, 8 Jan. 1999, Volume 283, presented the results of the study.

The researchers studied sixteen nonobese adults who underwent measures of both body composition and energy expenditure before and after eight weeks of supervised overfeeding by one thousand calories/day. As might be expected, the overfeeding led to a gain in weight. Indeed, the sixteen volunteers gained an average of ten pounds during the two-month study. However, weight gain varied between the volunteers from two pounds to almost sixteen pounds.

In their calculations to determine daily calorie expenditure, NEAT was looked at as one form of such expenditure. The researchers found that interindividual differences in BMR and postprandial thermogenesis did not correlate with the variability in fat gain. In addition, “volitional” exercise was stringently maintained at constant, low levels. However, the researchers noted that generally NEAT increased. The researchers further noted that those with a greater activation of NEAT tended to gain less weight than their counterparts. That is, as humans overeat, those with effective activation of NEAT may be able to dissipate the excess energy so that it is not available for storage as fat, whereas those with lesser degrees of NEAT activation may likely have greater fat gain and be predisposed to develop obesity. That being said, the research results suggest that efforts to enhance NEAT activation, perhaps through behavioral cues, may be a fruitful approach to the prevention of obesity.

As mentioned above, NEAT is the energy utilized for everything that is not sleeping, eating, or sports-like exercise. NEAT includes the energy expended walking to work, typing, performing yard work, undertaking agricultural tasks, and fidgeting. While it may be sensible to encourage energy expenditure (i.e., burn calories) through favoring the stairs over the elevator, gardening, walking to work, and so forth, such activities may be impractical to include during a large portion of a sedentary workday.

Accordingly, what is needed is a device that encourages an individual to enhance NEAT activation in everyday life, even if the individual is largely sedentary. Such a device may be utilized in combination with a weight management method for controlling an individual's weight, or for facilitating weight loss.

### SUMMARY OF THE INVENTION

Accordingly, it is an advantage of the present invention that an activity monitoring device is provided for encouraging an individual to enhance NEAT activation.

It is another advantage of the present invention that an activity monitoring device is provided that alerts an individual to increase their activity level.

Another advantage of the present invention is that a weight management method is provided that utilizes the activity monitoring device to encourage an individual to fidget when an increase in activity level is desirable.

The above and other advantages of the present invention are carried out in one form by an activity monitoring device that includes a movement sensor for producing a movement signal in response to an activity level of an individual. A timer is in communication with the movement sensor. The timer determines an interval during which the movement signal fails to exceed a movement intensity threshold. An indicator, in communication with the timer, alerts the individual when the interval exceeds a stationary interval threshold.

The above and other advantages of the present invention are carried out in another form by a weight management method that utilizes an activity monitoring device worn by an individual. The method calls for producing, at the activity monitoring device, a movement signal in response to an activity level of the individual and determining an interval during which the movement signal fails to exceed a movement intensity threshold. The activity monitoring devices alerts the individual when the interval exceeds a stationary interval threshold. In response to the alerting operation, the activity level of the individual is increased.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 shows a perspective view of an activity monitoring device in accordance with a preferred embodiment of the present invention;

FIG. 2 shows a side view of the activity monitoring device of FIG. 1;

FIG. 3 shows a block diagram of the activity monitoring device of FIG. 1;

FIG. 4 shows a table of menu items and an exemplary presentation of those menu items that may be provided on a display of the activity monitoring device;

FIG. 5 shows a flow chart of a weight management process utilizing the activity monitoring device of FIG. 1;

FIG. 6 shows a flow chart exemplifying an activity monitoring device operation process;

FIG. 7 shows an exemplary graph of signal strength of a movement signal versus time of a movement signal produced by the activity monitoring device; and

FIG. 8 shows an exemplary graph of a timer status versus time for a total movement timer included in the activity monitoring device.

### DETAILED DESCRIPTION OF THE PREFERRED EMBODIMENTS

As discussed in great detail above, non-exercise activity thermogenesis (NEAT) is the energy expended for everything that is not sleeping, eating, or sports-like exercise. NEAT includes, among other activities, the energy expended fidgeting. The present invention encourages an individual to enhance NEAT activation, and subsequently manage their weight, by alerting an individual to increase their activity level.

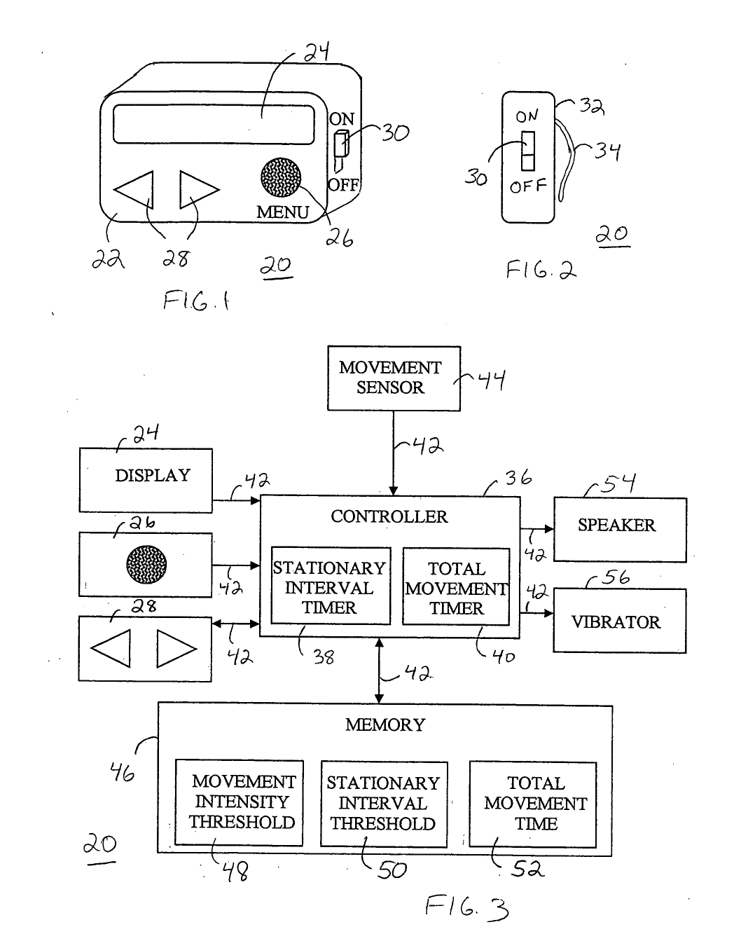

Referring to FIGS. 1-2, FIG. 1 shows a perspective view of an activity monitoring device 20 in accordance with a preferred embodiment of the present invention, and FIG. 2 shows a side view of activity monitoring device 20. Device 20 is an electronic monitoring device worn by an individual that on a real-time basis evaluates the individual's activity level (i.e., amount of fidgeting). When “still”, an individual wearing device 20 is alerted to this by an indicator. Once alerted, it is incumbent upon the individual to increase his or her activity level, i.e., to begin fidgeting, or to increase their intensity of movement. In this manner, an individual may increase energy expenditure, thus burning more calories, and subsequently better managing their weight.

A front face 22 of activity monitoring device 20 includes a display 24, a menu select button 26, and scroll buttons 28. An on/off switch 30 is located on the side of device 20. In a preferred embodiment, user interaction with activity monitoring device 20 is carried out utilizing display 24, menu select button 26, and scroll buttons 28 (as will be discussed below). A back side 32 of activity monitoring device 20 includes a clip 34. Activity monitoring device 20 is a hand-held device, and clip 34 readily attaches to an individual's clothing, such as on his or her waistband, after device 20 has been configured for operation.

Activity monitoring device 20 is shown having display 24, menu select button 26, scroll buttons 28, and On/Off switch 30 for simplicity of illustration. It should become apparent to those skilled in the art that the present invention may be adapted to include other configurations of buttons, toggle switches, touch-sensitive display screens, light emitting diodes (LEDs) mounted within a silkscreened instruction label, and the like. In addition, activity monitoring device 20 is shown with clip 34 for simplicity of illustration. It should be readily apparent, that other elements may be employed to otherwise attach device 20 to the individual. For example, device 20 may include straps (not shown) in place of clip 34 so that device 20 may be adjusted to fit around an ankle, leg, thigh, wrist, upper arm, and so forth. Alternatively, device 20 may be worn on a chain as a pendant, or device 20 may be carried within the user's pocket.

FIG. 3 shows a block diagram of activity monitoring device 20. In general, activity monitoring device 20 includes a controller 36 which executes program code (not shown) to generally control the operation of device 20. Controller 36 further maintains a stationary interval timer 38 and a total movement timer 40.

Controller 36 is in communication with external components via input/output (I/O) lines 42. These external components include display 24 and input elements, in the form of menu select button 26 and scroll buttons 28. Controller 36 is further in communication, via I/O lines 42, with a movement sensor 44 and memory 46 for retaining a movement intensity threshold 48, a stationary interval threshold 50, and a total movement time 52. Indicators, in the form of a speaker 54 and a vibrator 56 are further in communication with controller 36.

In an exemplary embodiment, movement sensor 44 is a three-axis tilt and vibration sensor that changes state when subjected to motion or vibration. As such, movement sensor 44 can readily detect minor movement, i.e., fidgeting, of an individual in three dimensions. Those skilled in the art will recognize, however, that there are a variety of sensors that may be employed to detect three dimensional movement, i.e., orientation, tilt, and gross motion. In addition, those skilled in the art will recognize that there are a variety of indicators that may be employed in combination with, or as an alternative to, speaker 54 and vibrator 56 to alert an individual to increase his or her activity level. Other such indicators may include, for example, text provided on display 24, light emitting diodes, and such.

In general, controller 36, executing the program code, periodically queries movement sensor 44 for a movement signal (discussed below) indicative of an activity level of the individual currently wearing activity monitoring device 20. Controller 36 compares the magnitude of the movement signal with movement intensity threshold 48. When the magnitude of the movement signal exceeds movement intensity threshold 48, controller 36 accrues the duration on total movement timer 40. The duration may subsequently be retained as total movement time 52 in memory 46.

When the magnitude of the movement signal fails to exceed movement intensity threshold value 48, controller 36 presumes that activity monitoring device 20 is “still” or “stationary.” Accordingly, controller 36 starts stationary interval timer 38. Controller 36 then continues to query movement sensor 44. When a stationary interval determined at stationary interval timer 38 exceeds stationary interval threshold 50 during which the movement signal fails to exceed movement intensity threshold value 48, controller 36 signals the indicators, i.e., speaker 54 and/or vibrator 56, to emit an alerting signal to alert the user of this situation. In response to the alerting signal, the user may increase his or her activity level, thus expending more energy and burning more calories. As such, activity monitoring device plays a role in behavior modification by alerting an individual when he or she is being unacceptably sedentary so that he or she may increase their activity level.

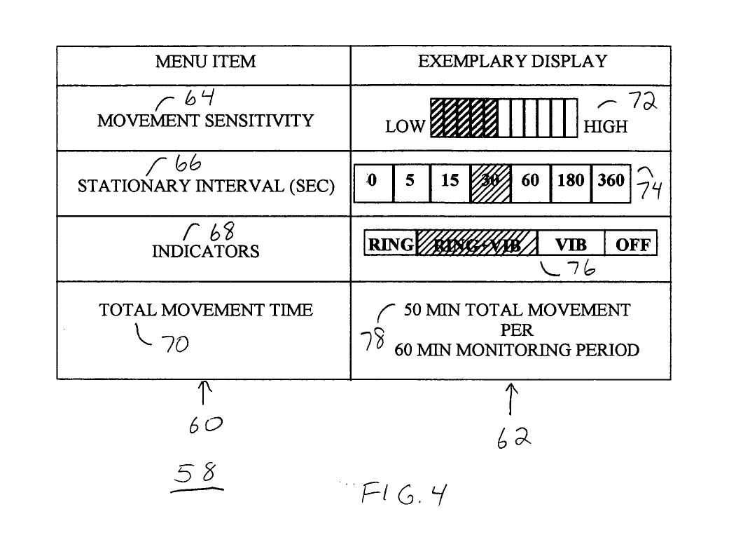

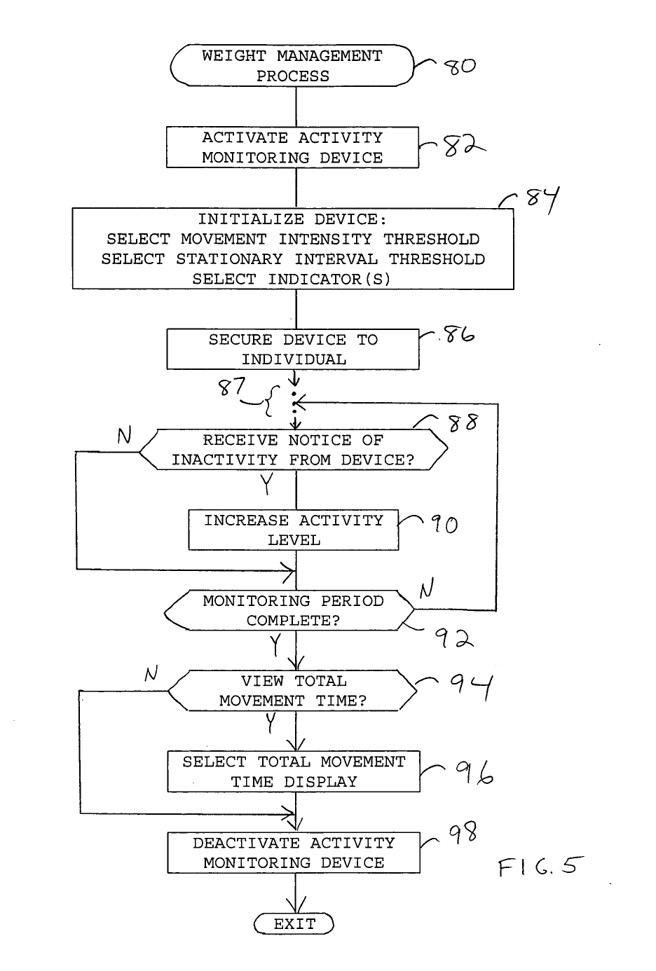

FIG. 4 shows a table 58 of a menu items and an exemplary presentation of those menu items that may be provided on display 24 (FIG. 1) of activity monitoring device (FIG. 20) when one of the menu items is selected. In particular, table 58 includes a menu item field 60 associated with an exemplary display field 62.

In a preferred embodiment, activity monitoring device 20 is configured to enable the user to adjust movement intensity threshold 48 (FIG. 3) and stationary interval threshold 50 (FIG. 3). In addition, device 20 is configured to enable the user to select which, if any, indicators (speaker 54 and vibrator 56) may be utilized to alert the user when he or she is unacceptably sedentary. One of the elements of menu item field 60 may also provide the user with feedback as to how active they have been during a monitoring period.

As shown, menu item field 60 of table 58 includes a movement sensitivity menu item 64, a stationary interval menu item 66, an indicators menu item 68, and a total movement time menu item 70. These menu items may be presented on display 24 when menu select button 26 is first activated. The menu items may be concurrently presented on display 24. Alternatively, the user may scroll through the menu items utilizing scroll buttons 28. Those skilled in the art will recognize that there are numerous techniques for the presentation of information on a small screen display, such as display 24. As such, the present invention need not be limited to any single display technique.

When movement sensitivity menu item 64 is selected, a sensitivity grid 72, as shown in exemplary display field 62, may be presented in display 24 (FIG. 3). Movement sensitivity represents the sensitivity of activity monitoring device 20 to movement. Thus, the user may raise or lower the sensitivity of device 20 to movement utilizing scroll buttons 28. High movement sensitivity translates to a low value for movement intensity threshold 48. When movement intensity threshold 48 is set low, a low detectable activity level (little movement) will exceed threshold 48, thus indicating that the individual is acceptably active (i.e., not sedentary). Conversely, low movement sensitivity translates to a high value for movement intensity threshold 48. Accordingly, when movement intensity threshold 48 is set high, an individual will be required to maintain a greater activity level, in order to exceed and stay above threshold 48. Thus, the user may raise or lower the sensitivity of device 20 to movement, hence the magnitude of movement intensity threshold 48, utilizing scroll buttons 28.

When stationary interval menu item 66 is selected, an interval grid 74, as shown in exemplary display field 62, may be presented in display 24 (FIG. 3). Selection of stationary interval menu item 66 enables a user to select a magnitude of stationary interval threshold 50 (FIG. 3), i.e., the allowable amount of time that an individual may be stationary. Thus, when stationary interval threshold 50 is set to a greater time, i.e., one hundred eighty seconds, three hundred sixty seconds, and the like, the user can have greater intervals of inactivity than when stationary interval threshold 50 is set to less time, i.e., five seconds, fifteen seconds, and the like.

Consequently, the combined settings of movement sensitivity menu item 64 and stationary interval menu item 66 characterize the activity level that activity monitoring device 20 is expecting from the individual. In general, high movement sensitivity and a short stationary interval threshold 50 result in the requirement that the individual have a greater activity level to prevent activation of speaker 54 and/or vibrator 56 (FIG. 3), than the alternative, i.e. low movement sensitivity and a long stationary interval threshold 50.

It should be noted that in an alternative embodiment, stationary interval threshold 50 may be set to zero (0) seconds. As such, activation of speaker 54 and/or vibrator 56 is not related to any stationary interval measured by stationary interval timer 38 (FIG. 3). Rather, activation of speaker 54 and/or vibrator 56 depends solely upon the movement sensitivity setting, i.e., the magnitude of movement intensity threshold 48, and the individual's current activity level. Consequently, when stationary interval threshold 50 is set to zero seconds and the individual's activity level is such that a movement signal (discussed below) fails to exceed movement intensity threshold 48, the indicators (speaker 54 and/or vibrator 56) will immediately alert the individual to his or her inactivity.

When indicators menu item 68 is selected, an indicators grid 76, as shown in exemplary display field 62, may be presented in display 24 (FIG. 3). Selection of indicators menu item 68 enables a user to select which of the indicators desired to alert the individual to their inactivity. By way of example, since activity monitoring device 20 includes speaker 54 (FIG. 3), and vibrator 56 (FIG. 3), the user may select “RING”, “RING+VIB”, “VIB”, and “OFF.”

When total movement time menu item 70 is selected, movement status text 78, as shown in exemplary display field 62, may be presented in display 24 (FIG. 3). Movement status text 78 represents an accrued duration for which the movement signal produced by movement sensor 44 (FIG. 3) exceeds movement intensity threshold 48, as timed by total movement timer 40 (FIG. 3) and recorded in memory 46 (FIG. 3) as total movement time 52. For example, as shown, there was fifty minutes of total movement per a sixty minute monitoring period.

FIG. 5 shows a flow chart of a weight management process 80 utilizing activity monitoring device 20 (FIG. 1). Weight management process 80 is provided to illustrate how an individual may utilize device 20 to enhance NEAT activation to manage their weight. However, it should be understood that device 20 may alternatively be utilized during a monitoring period that involves “volitional” exercise. In such a situation, the user may appropriately set movement intensity threshold 48 (FIG. 3) and stationary interval threshold 50 (FIG. 3) so that device 20 expects an intense activity level and short intervals during which the individual is allowed to be stationary. Of course, weight management process 80 may also be coupled with controlled caloric intake, exercise, medications, and even surgery, depending upon the degree of desired weight management or loss.

Process 80 begins with a task 82. At task 82, a user activates activity monitoring device 20 (FIG. 1). Next, at a task 84, device 20 is initialized. Initialization task 84 may be performed by the user, a health care provider, a dietician, or weight management consultant. At task 84, movement intensity threshold 48 (FIG. 3) is selected, via movement sensitivity menu item 64 (FIG. 4). In addition, stationary interval threshold 50 is selected, via stationary interval menu item 66 (FIG. 4), and indicators are selected via indicators menu item 68 (FIG. 4).

Next a task 86, is performed to secure device 20 (FIG. 1) to the individual. For example, in the embodiment shown in FIG. 1, clip 34 is attached to the user's waistband. Once device 20 is secured to the individual, the individual carries out his or her everyday tasks, as indicated by ellipsis 87. In the meantime, device 20 is monitoring the user's activity level and determining whether the user's activity level has dropped for a sufficient period of time that he or should be alerted to this inactivity.

Accordingly, a query task 88 determines whether the individual receives notice of inactivity from device 20. This notice of inactivity may be an alarm sounded at speaker 54 (FIG. 3), a vibration produced at vibrator 56 (FIG. 3), or both.

When a notice is received at query task 88, process control proceeds to a task 90. At task 90, it is incumbent upon the user to increase his or her activity level. The user may achieve this increase in activity level by fidgeting, walking, stretching, or otherwise moving about.

Following task 90, or when query task 88 determines that a notice of inactivity has not been received, a query task 92 is performed. At query task 92, a determination is made as to whether a monitoring period is complete. The monitoring period may be any duration selected by the user, a health care provider, a dietician, weight management consultant, and the like. The monitoring period could be, for example, an eight hour work day, sixteen hours, or a twenty-four hour period.

When the monitoring period is not complete at query task 92, process 80 loops back to ellipsis 87, symbolizing the user's continuation of everyday activity. When the monitoring period is complete, process 80 continues with a query task 94.

At query task 94, a determination is made as to whether the user wishes to view total movement time 52 (FIG. 3). When the user wishes to view total movement time 52, the user selects total movement time menu item 70 (FIG. 4) as discussed above. Total movement time 52 is subsequently presented on display 24 (FIG. 1) at a next task 96.

Following task 96 or when the user does not wish to view total movement time 52, as determined at query task 94, a task 98 is performed. At task 98, the user deactivates activity monitoring device 20 (FIG. 1).

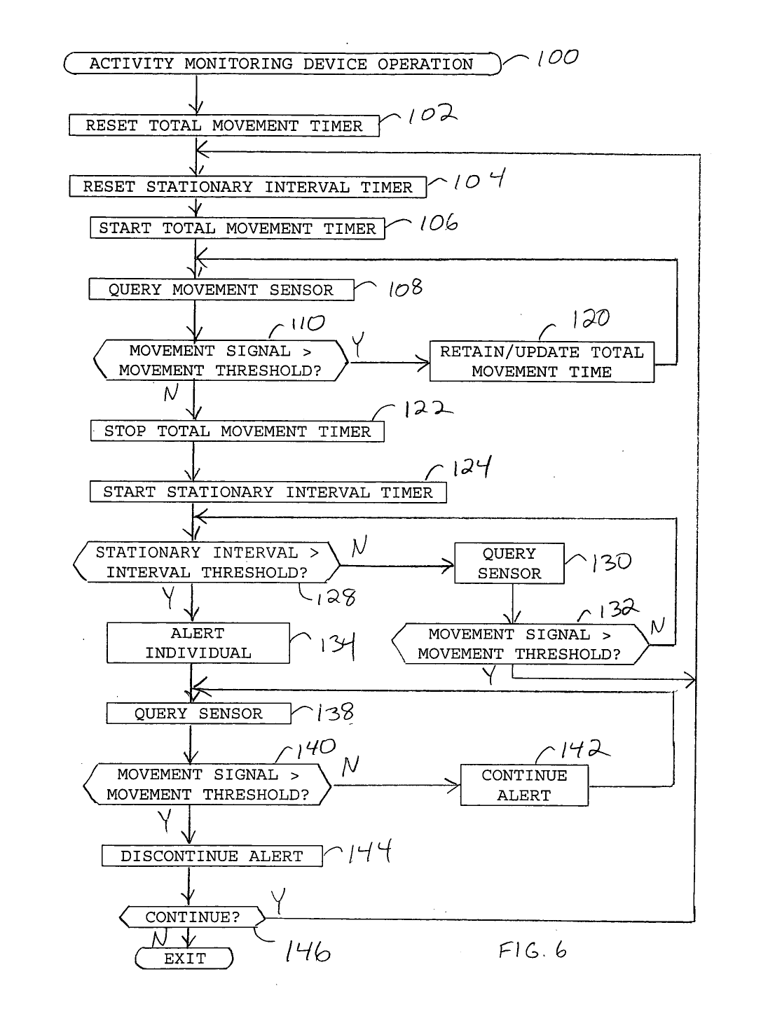

FIG. 6 shows a flow chart exemplifying an activity monitoring device operation process 100. Process 100 is provided to demonstrate the functionality of activity monitoring device 20 for encouraging an individual to enhance his or her activity level.

Process 100 begins with a task 102. Task 102 is executed within device 20 in response to activate operation 82 (FIG. 5) of weight management process 80 (FIG. 5). At task 102, total movement timer 40 (FIG. 3) is reset. That is, total movement timer 40 is set to zero so that timer 40 can accrue, or count total movement time 52 for this particular monitoring period.

Next, a task 104 is performed. At task 104, stationary interval timer 38 (FIG. 3) is also reset. That is, stationary interval timer is set to zero.

Following task 104, a task 106 is performed to start total movement timer 40 and begin accruing total movement time 52. Total movement timer 40 may automatically start immediately after zeroing task 104 or after a short interval, thus allowing a movement signal produced at movement sensor 44 (FIG. 3) to stabilize. Of course, those skilled in the art may recognize that task 106 need not be performed during a first iteration of process 100. Rather, during a first pass, process execution may proceed from reset tasks 102 and 104 directly to a task 108.

As such, following task 106, or alternatively, following task 104, movement sensor 44 (FIG. 3) is queried by controller 36 (FIG. 3) at task 108.

A query task 110 is performed in connection with task 108. Query task 110 determines whether the movement signal received at query task 108 is greater than movement intensity threshold 48 (FIG. 3).

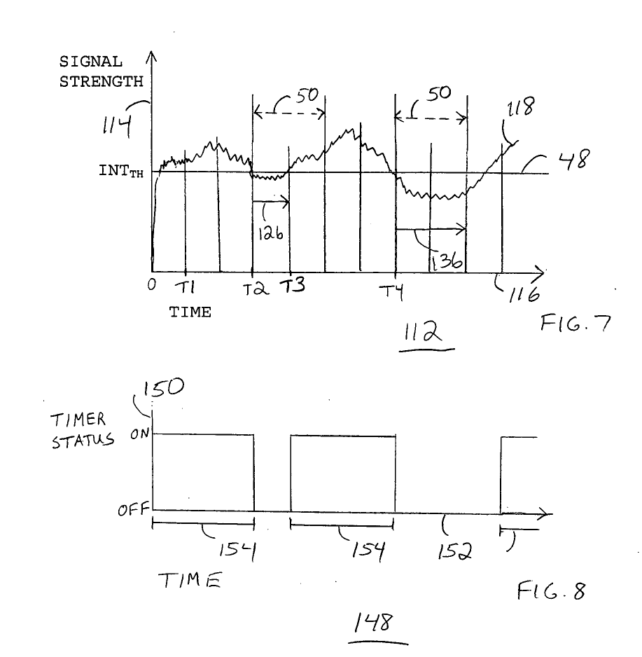

Referring to FIG. 7 in connection with query task 110, FIG. 7 shows an exemplary graph 112 of signal strength 114 versus time 116 of a movement signal 118 produced by movement sensor 44 (FIG. 4) of activity monitoring device 20 (FIG. 1). Graph 112 also shows movement intensity threshold (INT_{TH}) 48.

As shown, at a query time, T1, a magnitude of movement signal 118 exceeds intensity threshold 48. When query task 112 determines that movement signal 118 is greater than intensity threshold 48, process control proceeds to a task 120. At task 120, total movement time 52 is retained and otherwise updated. That is, total movement timer 40 (FIG. 3) is accruing time. This total movement time 52 may be written to memory 46 (FIG. 3). Following task 120, process control loops back to task 108 to again query movement sensor 44.

With continued reference to FIGS. 6-7, in response to querying movement sensor 44 at task 108, when query task 110 determines that movement signal 118 fails to exceed movement intensity threshold 48, process 100 proceeds to a task 122.

As shown, at a query time, T2, movement signal 118 fails to exceed intensity threshold 48. Accordingly, task 122 is performed so that total movement timer 40 is stopped, although total movement timer 40 is not reset, i.e., zeroed.

A task 124 is performed in connection with task 122. At task 124, stationary interval timer 38 (FIG. 3) is started to beginning timing a stationary interval 126, illustrated in graph 112.

Next, a query task 128 monitors stationary interval 126 currently being measured on stationary interval timer 38 and compares it against stationary interval threshold 50. For purposes of comparison, stationary interval threshold 50 is superimposed on graph 112 beginning at query time, T2. Query task 128 determines whether stationary interval 126 exceeds stationary interval threshold 50. When stationary interval 126 fails to exceed threshold 50, process 100 proceeds to a task 130.

At task 130, movement sensor 44 (FIG. 3) is queried. A query task 132 performed in cooperation with task 130 determines whether movement signal 118 exceeds movement intensity threshold 48. When movement signal 118 now exceeds movement intensity threshold 48, program control loops back to task 104. As such, stationary interval timer 38 is stopped and reset, i.e., zeroed, total movement timer 40 is restarted at task 106, and device 20 continues to monitor movement signal 118. Accordingly, query task 132 determines whether the individual wearing device 20 has spontaneously increased their activity level, for example, started walking, changed physical positions, started fidgeting, and so forth. This situation is exemplified in graph 112 in which movement sensor 44 is queried at query time, T3. Movement signal 118 now exceeds movement intensity threshold 48, while stationary interval 126 remains less than stationary interval threshold 50.

However, at query task 132, when movement signal 118 continues to remain less than movement intensity threshold 48, program control loops back to query task 128 to compare stationary interval 126 against stationary interval threshold 50. Consequently, while movement signal 118 is less than movement intensity threshold 48 and stationary interval 126 has not yet exceeded stationary interval threshold 50, device 20 merely continues to monitor the duration of inactivity.

When query task 128 determines that the stationary interval now approximates or exceeds stationary interval threshold 50, process 100 proceeds to a task 134. This situation is exemplified in graph 112 in which a stationary interval 136 measured a query time, T4, approximates or exceeds stationary interval threshold 50 at a query time T5 and a magnitude of movement signal 118 has remained below movement intensity threshold 48.

At task 134, device 20 alerts the individual by, for example, sounding a tone from speaker 54 (FIG. 3) and/or actuating vibrator 56 (FIG. 3. Alerting task 134 is intended to cue an individual to modify his or her behavior, i.e., begin to fidget, so as to enhance NEAT activation and burn additional calories. It is then incumbent upon the individual to increase his or her activity level, by fidgeting, moving around, taking a walk, and so forth.

In response to alerting task 134, device 20 (FIG. 1) continues to query movement sensor 44 at a task 138. A query task 140, performed in cooperation with task 138 determines whether movement signal 118 now exceeds movement intensity threshold 48. While movement signal 118 continues to remain below movement intensity threshold 48, a task 142 is performed. At task 142, device 20 continues to alert the individual. Process control then loops back to task 138 to continue monitoring movement sensor 44 and to compare movement signal 118 against movement intensity threshold 48.

However, when query task 140 determines that movement signal 118 now exceeds movement intensity threshold 48, process 100 proceeds to a task 144. At task 144, the alert signals are discontinued.

Following task 144, a query task 146 determines whether the activity level monitoring is to continue. Discontinuance of activity monitoring corresponds with query task 92 (FIG. 5) of weight management process 80 (FIG. 5) in which the individual determines whether the monitoring period is complete. When activity level monitoring is to be discontinued, process 100 exits. However, when activity level monitoring is to continue, process 100 loops back to task 104. As such, stationary interval timer 38 is stopped and reset, i.e., zeroed, total movement timer 40 is restarted at task 106, and device 20 continues to monitor movement signal 118.

FIG. 8 shows an exemplary graph 148 of a timer status 150 versus time 152 for total movement timer 40 (FIG. 3) of activity monitoring device 20 (FIG. 1). As shown, timer status 150 can be in one of two states, ‘on’ or ‘off’. For each episode in which timer status 150 is in the ‘on’ state, total movement timer 40 measures a movement duration 154. Consequently, total movement time 52 (FIG. 3) is a summation of these movement durations 154 for a particular monitoring period.

Although the use of activity monitoring device 20 is described in connection with weight management and alerting an individual when he or she should increase their activity level, the indicators, speaker 54 and vibrator 56, may be selectively turned off, as shown in indicators grid 76. When such is the case, the individual will not be alerted to their inactivity, but will still be able to accrue total movement time 52. This accrued total movement time 52 can then be studied to determine how and when the individual might want to modify their physical activity.

In summary, the present invention teaches of an activity monitoring device for encouraging an individual to enhance nonexercise activity thermogenesis (NEAT) activation. The activity monitoring device alerts an individual when their activity level has dropped below a movement intensity threshold for the entirety of a stationary interval threshold. Thus, the activity monitoring device can be utilized to encourage an individual to fidget when an increase in activity level is desirable.

Although the preferred embodiments of the invention have been illustrated and described in detail, it will be readily apparent to those skilled in the art that various modifications may be made therein without departing from the spirit of the invention or from the scope of the appended claims.

What is claimed is:

1. An activity monitoring device comprising:
a movement sensor for producing a movement signal in response to an activity level of an individual;
a timer in communication with said movement sensor, said timer determining an interval during which said movement signal fails to exceed a movement intensity threshold; and
an indicator in communication with said timer, said indicator alerting said individual when said interval exceeds a stationary interval threshold.

2. A device as claimed in claim 1 wherein said movement sensor is a tilt and vibration sensor.

3. A device as claimed in claim 1 wherein said timer is a first timer, and said device further comprises a second timer in communication with said movement sensor, said second timer measuring a duration for which said movement signal exceeds said movement intensity threshold.

4. A device as claimed in claim 3 wherein said second timer accrues a total movement time of a plurality of movement durations for a monitoring period, said duration being one of said plurality of movement durations.

5. A device as claimed in claim 4 further comprising a memory element in association with said second timer for retaining said total movement time.

6. A device as claimed in claim 4 further comprising a display in communication with said second timer for presenting said total movement time to said individual.

7. A device as claimed in claim 1 wherein said indicator is an audio alarm.

8. A device as claimed in claim 1 wherein said indicator is a vibrator element.

9. A device as claimed in claim 1 wherein said indicator is a display.

10. A device as claimed in claim 1 further comprising an input element in communication with said sensor for selecting a magnitude of said movement intensity threshold.

11. A device as claimed in claim 1 further comprising an input element in communication with said timer for selecting a magnitude of said stationary interval threshold.

12. A device as claimed in claim 1 wherein said device is configured to be worn by said individual.

13. A weight management method utilizing an activity monitoring device worn by an individual comprising:
producing, at said activity monitoring device, a movement signal in response to an activity level of said individual;
determining, at said activity monitoring device, an interval during which said movement signal fails to exceed a movement intensity threshold;
alerting said individual, from said activity monitoring device, when said interval exceeds a stationary interval threshold; and
increasing said activity level of said individual in response to said alerting operation.

14. A method as claimed in claim 13 further comprising discontinuing said alerting operation when said movement signal exceeds said movement intensity threshold.

15. A method as claimed in claim 13 further comprising:
measuring a plurality of movement durations for which said movement signal exceeds said movement intensity threshold;
accruing a total movement time of said plurality of movement durations for a monitoring period; and
presenting said total movement time to said individual.

16. A method as claimed in claim 13 further comprising selecting a magnitude of said movement intensity threshold.

17. A method as claimed in claim 13 further comprising selecting a magnitude of said stationary interval threshold.

18. An activity monitoring device comprising:
a movement sensor for producing a movement signal in response to an activity level of an individual;
a timer in communication with said movement sensor, said timer measuring a duration for which said movement signal exceeds a movement intensity threshold; and
a memory element in association with said timer for retaining a total movement time of a plurality of movement durations for a monitoring period, said duration being one of said plurality of movement durations.

19. A device as claimed in claim 18 further comprising a display in communication with said timer for presenting said total movement time to said individual.

20. A device as claimed in claim 18 further comprising an input element in communication with said movement sensor for selecting a magnitude of said movement intensity threshold.

21. An activity monitoring device comprising:
a movement sensor for producing a movement signal in response to an activity level of an individual; and
an indicator in communication with said movement sensor, said indicator alerting said individual when said movement signal fails to exceed a movement intensity threshold.

22. A device as claimed in claim 21 further comprising an input element in communication with said movement sensor for selecting a magnitude of said movement intensity threshold.

23. A device as claimed in claim 21 wherein said device further comprises a memory element in communication with said movement sensor, for accruing a plurality of movement durations for which said movement signal exceeds said movement intensity threshold.

24. A device as claimed in claim 23 further comprising a display in communication with said memory element for presenting a total movement time of said movement durations to said individual.
